(19) United States
(12) Patent Application Publication
(10) Pub. No.: US 2024/0029861 A1
(43) Pub. Date: Jan. 25, 2024

(54) HEALTH MANAGEMENT SUPPORT SYSTEM, ITS CONTROL METHOD, AND CONTROL PROGRAM

(71) Applicant: TOYOTA JIDOSHA KABUSHIKI KAISHA, Aichi-ken (JP)

(72) Inventors: Issei NAKASHIMA, Tokyo-to (JP); Toru MIYAGAWA, Seto-shi (JP); Hideo HASEGAWA, Nagoya-shi (JP); Yusuke KINOSHITA, Tokyo-to (JP); Hiroki SATO, Tokyo-to (JP); Hiroaki KAMEI, Tokyo-to (JP); Maika HASHIMOTO, Urayasu-shi (JP); Manabu YAMAMOTO, Toyota-shi (JP)

(21) Appl. No.: 18/357,156

(22) Filed: Jul. 24, 2023

(30) Foreign Application Priority Data
Jul. 25, 2022 (JP) 2022-117832

(51) Int. Cl. G16H 20/60; G16H 50/70

(52) U.S. Cl. CPC G16H 20/60; G16H 50/70

(57) ABSTRACT

A health management support system includes: a first acquisition unit configured to acquire information about each of members of a specific group to which a user belongs, including his/her health condition; a second acquisition unit configured to acquire information about a food purchased by the user; an inference unit configured to infer, when an abnormality is detected in a health condition of any one of a plurality of members belonging to the specific group, as to which food has caused the abnormality in the health condition of the member from a history of foods purchased by the user; and a notification unit configured to notify the user that the food inferred by the inference unit may have caused the abnormality in the health condition of the member.

### CROSS REFERENCE TO RELATED APPLICATIONS

This application is based upon and claims the benefit of priority from Japanese patent application No. 2022-117832, filed on Jul. 25, 2022, the disclosure of which is incorporated herein in its entirety by reference.

### BACKGROUND

The present disclosure relates to a health management support system, its control method, and a control program.

Japanese Unexamined Patent Application Publication No. 2009-276377 discloses a method for providing an advertisement, including a step of calculating a scheduled date of consumption for a commodity based on a date of purchase thereof and information about a person who purchased the commodity, in which in the step of calculating the scheduled date for consumption of the commodity, the number of days of consumption associated with the same commodity as the purchased commodity and the same family structure as that of the purchaser is read from accumulation means in which commodities, family structures, and the numbers of days of consumption are stored in association with each other, and the scheduled date of consumption for the commodity is calculated by adding the read number of days of consumption to the date of purchase.

### SUMMARY

However, in Japanese Unexamined Patent Application Publication No. 2009-276377, there is a problem that when an abnormality occurs not only in the health condition of a purchaser of foods but also in the health condition of any one of the other members of the family of the purchaser of the foods, the food that has caused the abnormality cannot be inferred, so that the family cannot improve their eating habits.

The present disclosure has been made in view of the above-described circumstances, and an object thereof is to provide a health management support system, its control method, and a control program capable of urging members of a group to which a purchaser of foods belongs to efficiently improve their eating habits.

A health management support system according to an aspect of the present disclosure includes: a first acquisition unit configured to acquire information about each of members of a specific group to which a user belongs, including his/her health condition; a second acquisition unit configured to acquire information about a food purchased by the user; an inference unit configured to infer, when an abnormality is detected in a health condition of any one of a plurality of members belonging to the specific group, as to which food has caused the abnormality in the health condition of the member from a history of foods purchased by the user; and a notification unit configured to notify the user that the food inferred by the inference unit may have caused the abnormality in the health condition of the member. In this health management support system, when an abnormality is detected in a health condition of a member of a specific group to which a user who has purchased foods belongs, the user is notified of a food that may have caused the abnormality in the health condition of that member, so that the member can efficiently improve his/her eating habits by referring to the content of the notification. Further, the member can determine the subsequent method for treating the abnormality by referring to the content of the notification.

The information about each of the members may include information about at least one of an age, a gender, a height, and a weight of the member. The accuracy of the inference as to which food has caused an abnormality in the health condition of any one of a plurality of members belonging to a specific group to which the user belongs can be improved by taking the gender, the age, and the like of each of the members belonging to the specific group, in addition to the health condition of each of the members, into consideration.

The information about each of the members may include information about a food that that member is recommended to refrain from ingesting.

The inference unit may: extract, from a first database in which combinations of foods and symptoms that are possibly caused by the foods are registered, at least one food corresponding to at least one symptom that is determined to be similar to that of the member for which the abnormality has been detected in the health condition, and infer, from the history of foods purchased by the user, that a food corresponding to the extracted food has caused the abnormality in the health condition of the member.

The health management support system may further include a selection unit configured to select, as an alternative food, a food as a substitute for the food that the inference unit has inferred has caused the abnormality in the health condition of the member, in which the notification unit may also notify the user of information about the alternative food.

A method for controlling a health management support system according to an aspect of the present disclosure includes: acquiring information about each of members of a specific group to which a user belongs, including his/her health condition; acquiring information about a food purchased by the user; inferring, when an abnormality is detected in a health condition of any one of a plurality of members belonging to the specific group, as to which food has caused the abnormality in the health condition of the member from a history of foods purchased by the user; and notifying the user that the inferred food may have caused the abnormality in the health condition of the member. In this method for controlling a health management support system, when an abnormality is detected in a health condition of a member of a specific group to which a user who has purchased foods belongs, the user is notified of a food that may have caused the abnormality in the health condition of that member, so that the member can efficiently improve his/her eating habits by referring to the content of the notification. Further, the member can determine the subsequent method for treating the abnormality by referring to the content of the notification.

A control program according to an aspect of the present disclosure causes a computer to perform: a process for acquiring information about each of members of a specific group to which a user belongs, including his/her health condition; a process for acquiring information about a food purchased by the user; a process for inferring, when an abnormality is detected in a health condition of any one of a plurality of members belonging to the specific group, as to which food has caused the abnormality in the health condition of the member from a history of foods purchased by the user; and a process for notifying the user that the inferred food may have caused the abnormality in the health condition of the member. In this control program, when an abnormality is detected in a health condition of a member of a specific group to which a user who has purchased foods belongs, the user is notified of a food that may have caused the abnormality in the health condition of that member, so that the member can efficiently improve his/her eating habits by referring to the content of the notification. Further, the member can determine the subsequent method for treating the abnormality by referring to the content of the notification.

According to the present disclosure, it is possible to provide a health management support system, its control method, and a control program capable of urging members of a group to which a purchaser of foods belongs to efficiently improve their eating habits.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a health management support system according to a first embodiment;

FIG. 2 is a block diagram showing an example of a specific configuration of a health management support apparatus provided in the health management support system shown in FIG. 1;

FIG. 3 is a block diagram showing a modified example of the health management support system shown in FIG. 1;

FIG. 4 is a block diagram showing an example of a specific configuration of a health management support apparatus provided in the health management support system shown in FIG. 3;

FIG. 5 is a flowchart showing operations performed by the health management support system shown in FIG. 1;

FIG. 6 shows an example of a specific group to which a user belongs and its members; and

FIG. 7 shows an example of a food purchase history.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

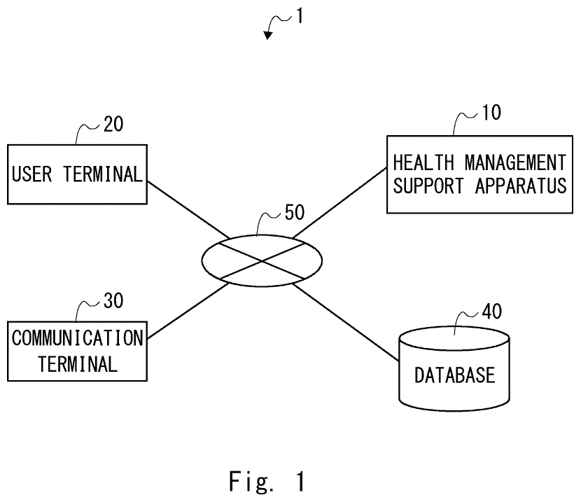

FIG. 1 is a block diagram showing an example of a configuration of a health management support system 1 according to a first embodiment. In the health management support system 1, when an abnormality is detected in the health condition of a member of a specific group to which a user who has purchased foods belongs, the user is notified of a food that may have caused the abnormality in the health condition of that member, so that the member can efficiently improve his/her eating habits by referring to the content of the notification. That is, the health management support system 1 can urge the members of the specific group to which the user who has purchased foods belongs to efficiently improve his/her eating habits. Details of the health management support system 1 and the like will be described hereinafter.

As shown in FIG. 1, the health management support system 1 includes a health management support apparatus 10, a user terminal 20, a communication terminal 30, a database 40, and a network 50. The health management support apparatus 10 can also be referred to as a health management support system. The health management support apparatus 10, the user terminal 20, the communication terminal 30, and the database 40 are configured so that they can communicate with each other through the wired or wireless network 50.

The user terminal 20 is a terminal owned by or temporarily assigned to a user U1 and capable of performing communication such as a PC (Personal Computer) terminal, a mobile terminal such as a smartphone or a tablet-type terminal, or a dedicated communication terminal provided for the health management support system. For example, the user U1 enters information about each of the members of a specific group to which the user U1 belongs into the user terminal 20 by operating an operation screen, a keyboard, or the like of the user terminal 20. The user terminal 20 receives the information about each of the members of the specific group to which the user U1 belongs, entered by the operation performed by the user U1, and transmits the received information to the health management support apparatus 10 through the network 50.

Note that the specific group to which the user U1 belongs is, for example, the family of the user U1 or the residents of a shared house where the user U1 lives.

The information about each of the members of the specific group to which the user U1 belongs includes at least information about the health condition of that member. The information about the health condition of each member includes, for example, information as to whether the health condition of that member is good or not, and when the member is ill, information about the progress of the disease. Note that a result of a health examination of each member may be used as the information about the health condition of that member.

Further, the information about each member may include at least one of the age, the gender, the height, and the weight of that member. In this way, it is possible to improve the accuracy of the inference as to which food has caused an abnormality of the health condition of any one of the members, made by the health management support apparatus 10 (which will be described later).

Further, the information about each member may include information about a food that that member is recommended to refrain from ingesting. As the foods that the member is recommended to refrain from ingesting, foods that are roughly classified, such as those classified as being fish, meat, vegetables, and the like, may be designated, or foods that are more precisely classified, such as those classified as being pike, salmon, eel, and the like, may be designated. More specifically, as the foods that the member is recommended to refrain from ingesting, for example, eggs, milk, wheat, or buckwheat, which may cause food allergies, may be designated. Alternatively or additionally, fatty foods such as fatty tuna, which infants are recommended to refrain from ingesting, may be designated. In this way, it is possible to improve the accuracy of the inference as to which food has caused an abnormality of the health condition of any one of the members, made by the health management support apparatus 10 (which will be described later).

Note that although an example case where the user terminal 20 receives information about each of the members of a specific group to which a user U1 belongs, and transmits the received information to the health management support apparatus 10 through the network 50 has been described in this embodiment, the present disclosure is not limited to this example. For example, a communication terminal (not shown) installed in a hospital may receive a result or the like of a health examination or a medical examination of each member, and transmit the received result or the like to the health management support apparatus 10 through the network 50. Alternatively, a wearable terminal (not shown) worn by each member may transmit vital data such as a heart rate and a pulse rate of that member to the health management support apparatus 10 through the network 50.

The communication terminal 30 is a terminal capable of performing communication, installed in a cash register or the like of a store where foods are sold, such as a PC terminal, a mobile terminal such as a tablet-type terminal, or a dedicated communication terminal provided for the health management support system. The communication terminal 30 reads information about foods purchased by the user U1 who has visited the store, entered by an operation performed by a store clerk or the user U1, and transmits the received information to the health management support apparatus 10 through the network 50.

Note that although an example case where the health management support system 1 includes one communication terminal 30 installed in one store has been described in this embodiment, the present disclosure is not limited to this example. The health management support system 1 may include a plurality of communication terminals 30 installed in one store, or may include a plurality of communication terminals 30 installed in a plurality of stores, respectively. In that case, each communication terminal 30 receives information about foods purchased by the user U1 and transmits the received information to the health management support apparatus 10 through the network 50.

Further, although an example case where the communication terminal 30 receives information about foods purchased by the user U1 and transmits the received information to the health management support apparatus 10 through the network 50 has been described in this embodiment, the present disclosure is not limited to this example. For example, the user terminal 20 may receive information about foods purchased by the user U1, entered by an operation performed by the user U1, and transmit the received information to the health management support apparatus 10 through the network 50.

When an abnormality is detected in the health condition of any one of a plurality of members of a specific group to which the user U1 belongs, the health management support apparatus 10 infers a food that has caused the abnormality in the health condition of that member from a history of foods purchased by the user U1, and notifies the user U1 of the result of the inference.

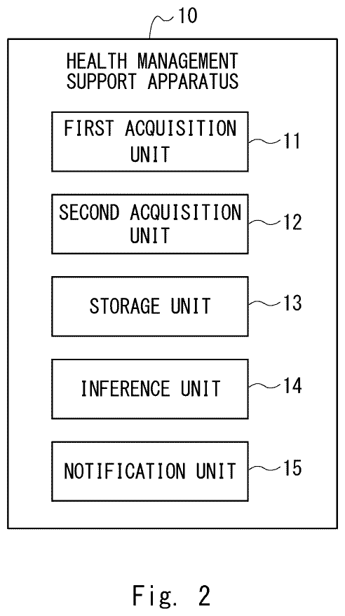

FIG. 2 is a block diagram showing an example of a configuration of the health management support apparatus 10. As shown in FIG. 2, the health management support apparatus 10 includes at least a first acquisition unit 11, a second acquisition unit 12, a storage unit 13, an inference unit 14, and a notification unit 15.

The first acquisition unit 11 acquires information about each of the members of the specific group to which the user U1 belongs, transmitted from the user terminal 20 or the like.

The second acquisition unit 12 acquires information about foods purchased by the user U1, transmitted from the communication terminal 30 or the like attached to (or installed in) a cash register or the like of the store. The information acquired by the second acquisition unit 12 is stored in the storage unit 13 as a food purchase history of the user U1. Note that the storage unit 13 may be provided outside the health management support apparatus 10.

When an abnormality is detected in the health condition of any one of the plurality of members belonging to the specific group, the inference unit 14 infers, from the food purchase history of the user U1, as to which food has caused the abnormality in the health condition of that member. Note that the occurrence of an abnormality in the health condition of a member may be detected in the inference unit 14 based on a change in the health condition of the member that is periodically acquired, or by receiving information indicating that an abnormality has occurred in the health condition of any one of the members from the user terminal 20 or the like.

For example, when a symptom of a food allergy is detected in a member of which an abnormality has occurred in the health condition, the inference unit 14 extracts a food that has caused the food allergy from the food purchase history of the user U1 and infers the extracted food as the food that has caused the abnormality in the health condition of the member. Alternatively, when a symptom that is caused by the ingestion of a food that the member for which an abnormality has occurred in the health condition is recommended to refrain from ingesting is detected in that member, the inference unit 14 extracts the food that the member is recommended to refrain from ingesting from the food purchase history of the user U1 and infers the extracted food as the food that has caused the abnormality in the health condition of the member.

The estimation unit 14 will be described in a more detailed manner. For example, a plurality of combinations of foods and symptoms that may be caused by the foods are registered in the database 40. Firstly, the inference unit 14 extracts, from the database 40, at least one food corresponding to at least one symptom that is determined to be similar to the symptom of the member for which the abnormality has occurred in the health condition. After that, the inference unit 14 infers, among a plurality of foods listed in the food purchase history of the user U1, a food that matches the food extracted from the database 40 (i.e., the same food as the food extracted from the database 40) as the food that has caused the abnormality in the health condition of the member.

Note that the data registered in the database 40 is not limited to a plurality of combinations of foods and symptoms. For example, a plurality of combinations of foods, symptoms, and at least one of an age, a gender, a height and a weight may be registered therein. In this way, the inference unit 14 can easily narrow down the conditions, and thereby can improve the accuracy of the inference as to which food has caused the abnormality in the health condition of the member.

The notification unit 15 notifies the user U1 that the food inferred by the inference unit 14 may have caused the abnormality in the health condition of the member. The information of which the user U1 is to be notified by the notification unit 15 is, for example, transferred to the user terminal 20 through the network 50, and the user U1 is thereby notified thereof.

As described above, in the health management support system 1 according to this embodiment, when an abnormality is detected in the health condition of a member of a specific group to which a user who has purchased foods belongs, the user is notified of a food that may have caused the abnormality in the health condition of that member, and the member can efficiently improve his/her eating habits by referring to the content of the notification. Further, the member can determine the subsequent method for treating the abnormality by referring to the content of the notification. That is, the health management support system 1 can urge the members of the specific group to which the user who has purchased foods belongs to efficiently improve his/her eating habits.

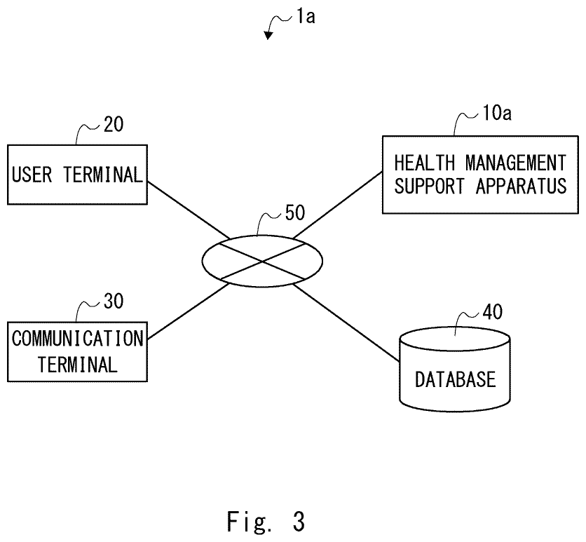

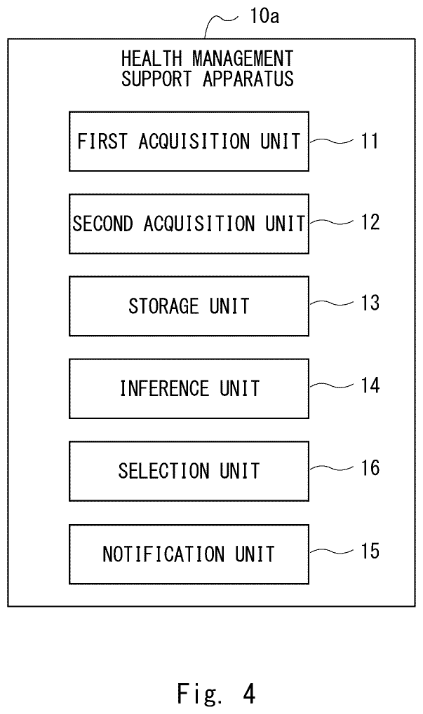

Note that the health management support apparatus 10 may be configured so as to propose, as an alternative food, a substitute food for the food inferred by the inference unit 14, i.e., the food that has caused the abnormality in the health condition of the member. This feature will be briefly described hereinafter with reference to FIGS. 3 and 4.

FIG. 3 is a block diagram showing a modified example of the health management support system 1 as a health management support system 1a. The health management support system 1a includes a health management support apparatus 10a in place of the health management support apparatus 10. FIG. 4 is a block diagram showing an example of a specific configuration of the health management support apparatus 10a. The health management support apparatus 10a further includes a selection unit 16 as compared with the health management support apparatus 10.

The selection unit 16 selects, as an alternative food, a substitute food for the food inferred by the inference unit 14, i.e., the food that has caused the abnormality in the health condition of the member. Then, the notification unit 15 notifies the user not only that the food inferred by the inference unit 14 may have caused the abnormality in the health condition of the member, but also of information about the alternative food selected by the selection unit 16. The rest of the configuration of the health management support system 1a is similar to that of the health management support system 1, and therefore the description thereof is omitted.

### (Operation of Health Management Support System 1)

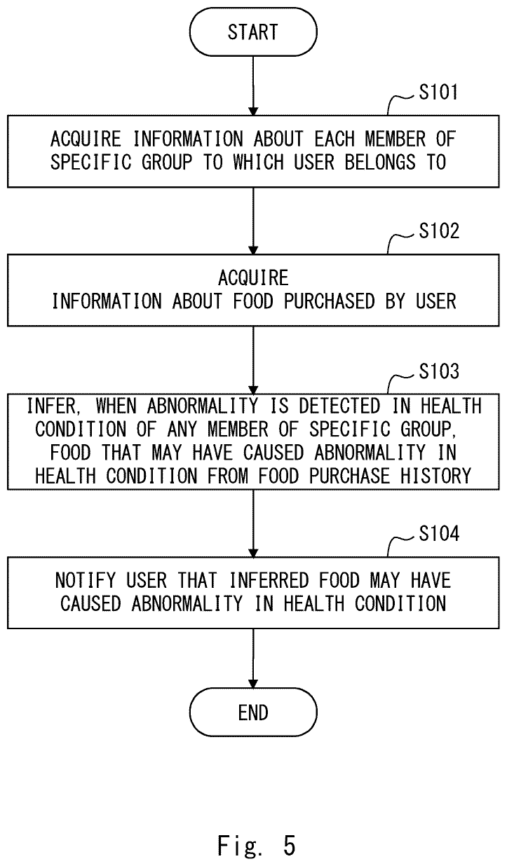

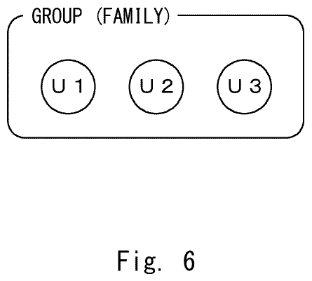

Next, operations performed by the health management support system 1 will be described with reference to FIG. 5. FIG. 5 is a flowchart showing operations performed by the health management support system 1. Note that in this embodiment, an example case where the group to which the user U1 belongs is the family of the user U1, consisting of members U1 to U3 (see FIG. 6) will be described. The user U1 and the member U1 are the same person.

Firstly, the user terminal 20 such as a smartphone receives information about each of the members of the specific group to which the user U1 belongs, entered by an operation performed by the user U1, and transmits the received information to the health management support apparatus 10 through the network 50.

For example, the user terminal 20 receives information about each of the members U1 to U3 of the family of the user U1, and transmits the received information to the health management support apparatus 10 through the network 50. The information about the members U1 to U3 includes at least information about the health conditions of the members U1 to U3, respectively. Note that as already described above, the apparatus or the like that receives and transmits information about each of the members U1 to U3 is not limited to the user terminal 20. That is a communication terminal installed in a hospital or a wearable terminal may receive information about each of the members U1 to U3 and transmit the received information to the health management support apparatus 10 through the network 50.

Further, the communication terminal 30 installed in a cash register or the like of a store receives information about foods purchased by the user U1 who has visited the store, entered by an operation performed by a store clerk or the user U1, and transmits the received information to the health management support apparatus through the network 50. Note that as already described above, the apparatus or the like that receives and transmits information about foods purchased by the user U1 is not limited to the communication terminal 30. That is, the user terminal 20 may receive information about foods purchased by the user U1, and transmit the received information to the health management support apparatus 10 through the network 50.

In the health management support apparatus 10, the first acquisition unit 11 acquires information about each of the members of the specific group to which the user U1 belongs, transmitted from the user terminal 20 or the like (Step S101). In this example, the first acquisition unit 11 acquires information about each of the members U1 to U3 of the family of the user U1.

Further, in the health management support apparatus 10, the second acquisition unit 12 acquires information about foods purchased by the user U1, transmitted from the communication terminal 30 or the like attached to (or installed in) a cash register or the like of a store (Step S102). The information acquired by the second acquisition unit 12 is stored in the storage unit 13 as a food purchase history of the user U1.

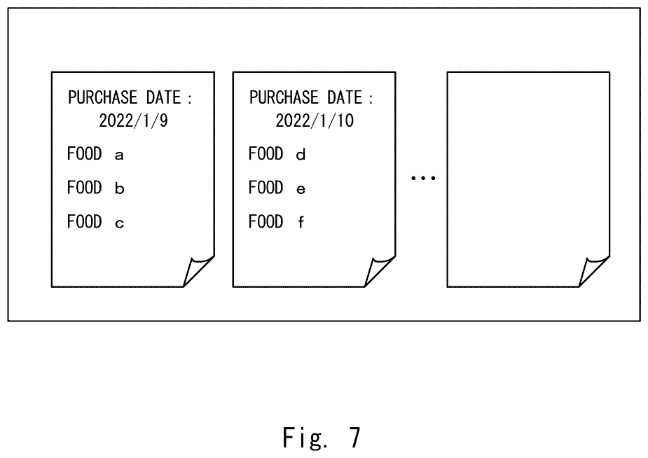

FIG. 7 shows an example of the food purchase history of the user U1. As shown in FIG. 7, foods a, b and c were purchased on Jan. 9, 2022, and foods d, e and f were purchased on Jan. 10, 2022.

After that, in the health management support apparatus 10, when an abnormality is detected in the health condition of any one of a plurality of members belonging to the specific group, the inference unit 14 infers a food that has caused the abnormality in the health condition of that member from the food purchase history of the user U1 (Step S103).

Specifically, firstly, the inference unit 14 extracts, from the database 40, at least one food corresponding to at least one symptom that is determined to be similar to the symptom of the member for which the abnormality has occurred in the health condition. After that, the inference unit 14 infers, among a plurality of foods listed in the food purchase history of the user U1, a food that matches the food extracted from the database 40 (i.e., the same food as the food extracted from the database 40) as the food that has caused the abnormality in the health condition of the member.

For example, when a symptom of a food allergy is detected in the member U2 (e.g., the wife of the user U1), the inference unit 14 extracts a food that may have caused the food allergy (e.g., the food a) from among the foods a to f listed in the food purchase history of the user U1, and infers the extracted food as the food that has caused the abnormality in the health condition of the member U2. Alternatively, when a symptom that is caused by the ingestion of a food that the member U3 (e.g., a child of the user U1) is recommended to refrain from ingesting is detected in the member U3, the inference unit 14 extracts a food that the member U3 is recommended to refrain from ingesting (e.g., the food d) from among the foods a to f listed in the food purchase history of the user U1, and infers the extracted food as the food that has caused the abnormality in the health condition of the member U3.

After that, the notification unit 15 notifies the user U1 that the food inferred by the inference unit 14 may have caused the abnormality in the health condition of the member (Step S104). The information of which the user U1 is to be notified by the notification unit 15 is, for example, transferred to the user terminal 20 through the network 50, and the user U1 is thereby notified thereof.

As described above, in the health management support system 1 according to this embodiment, when an abnormality is detected in the health condition of a member of a specific group to which a user who has purchased foods belongs, the user is notified of a food that may have caused the abnormality in the health condition of that member, and the member can efficiently improve his/her eating habits by referring to the content of the notification. Further, the member can determine the subsequent method for treating the abnormality by referring to the content of the notification. That is, the health management support system 1 can urge the members of the specific group to which the user who has purchased foods belongs to efficiently improve his/her eating habits.

Note that the present disclosure is not limited to the above-described embodiments, and they can be modified as appropriate without departing from the scope and spirit of the disclosure.

Further, in the present disclosure, some or all of the control processes performed in the health management support system 1 can be implemented by having a CPU (Central Processing Unit) execute a computer program.

In the aforementioned program includes instructions (or software codes) that, when loaded into a computer, cause the computer to perform at least one of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A health management support system comprising:
a first acquisition unit configured to acquire information about each of members of a specific group to which a user belongs, including his/her health condition;
a second acquisition unit configured to acquire information about a food purchased by the user;
an inference unit configured to infer, when an abnormality is detected in a health condition of any one of a plurality of members belonging to the specific group, as to which food has caused the abnormality in the health condition of the member from a history of foods purchased by the user; and
a notification unit configured to notify the user that the food inferred by the inference unit may have caused the abnormality in the health condition of the member.

2. The health management support system according to claim 1, wherein the information about each of the members includes information about at least one of an age, a gender, a height, and a weight of the member.

3. The health management support system according to claim 1, wherein the information about each of the members includes information about a food that that member is recommended to refrain from ingesting.

4. The health management support system according to claim 1, wherein
the inference unit:
extracts, from a first database in which combinations of foods and symptoms that are possibly caused by the foods are registered, at least one food corresponding to at least one symptom that is determined to be similar to that of the member for which the abnormality has been detected in the health condition, and
infers, from the history of foods purchased by the user, that a food corresponding to the extracted food has caused the abnormality in the health condition of the member.

5. The health management support system according to claim 1, further including a selection unit configured to select, as an alternative food, a food as a substitute for the food that the inference unit has inferred has caused the abnormality in the health condition of the member, wherein
the notification unit also notifies the user of information about the alternative food.

6. A method for controlling a health management support system, comprising:
acquiring information about each of members of a specific group to which a user belongs, including his/her health condition;
acquiring information about a food purchased by the user;
inferring, when an abnormality is detected in a health condition of any one of a plurality of members belonging to the specific group, as to which food has caused the abnormality in the health condition of the member from a history of foods purchased by the user; and
notifying the user that the inferred food may have caused the abnormality in the health condition of the member.

7. A non-transitory computer readable storage medium storing a control program for causing a computer to perform:
a process for acquiring information about each of members of a specific group to which a user belongs, including his/her health condition;
a process for acquiring information about a food purchased by the user;
a process for inferring, when an abnormality is detected in a health condition of any one of a plurality of members belonging to the specific group, as to which food has caused the abnormality in the health condition of the member from a history of foods purchased by the user; and
a process for notifying the user that the inferred food may have caused the abnormality in the health condition of the member.
